(19) United States
(12) Patent Application Publication
(10) Pub. No.: US 2016/0122276 A1
(43) Pub. Date: May 5, 2016

(54) PREPARATION OF C8-C22 ALKYL (METH)ACRYLATES

(71) Applicant: BASF SE, Ludwigshafen (DE)

(72) Inventors: Andrea MISSKE, Speyer (DE); Hannelore Schirra, Hockenheim (DE); Christoph Fleckenstein, Freigericht (DE); Martin Kaller, Mannheim (DE); Ulrik Stengel, Birkenau (DE); Mathieu Blanchot, Lambsheim (DE); Friederike Fleischhaker, Ludwigshafen (DE); Ritesh Nair, Heidelberg (DE)

(73) Assignee: BASF SE, Ludwigshafen (DE)

(21) Appl. No.: 14/883,803

(22) Filed: Oct. 15, 2015

Related U.S. Application Data
(60) Provisional application No. 62/075,282, filed on Nov. 5, 2014.

(30) Foreign Application Priority Data
Nov. 5, 2014 (DE) 10 2014 222 584.1

(51) Int. Cl. C07C 67/02

(52) U.S. Cl. CPC C07C 67/02

(57) ABSTRACT

The invention relates to a method of preparing a C₈-C₂₂ alkyl (meth)acrylate by transesterification of C₁-C₂ alkyl (meth)acrylate with a C₈-C₂₂ alkanol, said method comprising the steps of

(i) reacting C₁-C₂ alkyl (meth)acrylate with the C₈-C₂₂ alkanol in the presence of a particulate potassium phosphate heterogeneous catalyst and a stabilizer thus releasing C₁-C₂ alkanol,

(ii) continuously distilling off the azeotrope of C₁-C₂ alkyl (meth)acrylate and the C₁-C₂ alkanol,

wherein steps (i) and (ii) are carried out simultaneously until substantially all of the C₈-C₂₂ alkanol has reacted,

(iii) distilling off unconverted C₁-C₂ alkyl (meth)acrylate,

(iv) washing the C₈-C₂₂ alkyl (meth)acrylate-comprising product mixture obtained in steps (i) through (iii) with an aqueous phase to separate off the catalyst and the stabilizer from the product mixture with the aqueous phase and optionally adding a stabilizer,

(v) distilling off water from the product mixture,

wherein step (iii) may also be effected after step (iv) and together with step (v) and step (v) affords a product having a purity of >98 wt %.

The invention relates to a method of preparing C₈-C₂₂ alkyl (meth)acrylates by transesterification of C₁-C₂ alkyl (meth)acrylate with C₈-C₂₂ alkanols.

Polymers or copolymers prepared on the basis of branched C₈⁻C₂₂ (meth)acrylates are of considerable economic importance in the form of polymer dispersions. They are used, for example, as adhesives, lubricants, oil field chemicals and paints and as textile, leather and papermaking assistants. Hereinbelow, (meth)acrylic acid and (meth)acrylate are used as umbrella terms for acrylic acid/methacrylic acid and acrylate/methacrylate respectively.

The preparation of alkylated (meth)acrylates by acid-catalyzed esterification of (meth)acrylic acid with higher alkanols is described in WO 2002/050014 A1 and WO 2002/050015 A1 for example.

WO 2009/106550 describes a method of preparing (meth)acrylates from C₁₀ alkanols by esterification of (meth)acrylic acid with an isomer mixture of C₁₀ alkanols comprising 2-propylheptanol as the main isomer in the presence of an acidic catalyst, a polymerization inhibitor and a solvent which forms an azeotrope with water, wherein the esterification is carried out in a reactor equipped with a circulatory evaporator and the crude product obtained is purified by subsequent purifying distillation.

In the sole working example, acrylic acid is esterified with 2-propylheptanol. This comprises initially charging cyclohexane, 2-propylheptanol and hypophosphorous acid and adding to this mixture the stabilizer hydroquinone monomethyl ether (MEHQ), hypophosphorous acid, copper(II) chloride solution and acrylic acid. The mixture is heated under an air atmosphere, methanesulfonic acid is added and water is continuously separated off under reflux. The cooled-down reaction solution is washed with sodium chloride solution and aqueous sodium hydroxide solution. The cyclohexane phase is separated off and the solvent is removed under reduced pressure. 2-Propylheptyl acrylate is obtained in 97% yield and >95% purity. Once the purifying distillation has been carried out, 2-propylheptyl acrylate is obtained in >99% purity.

DE 10 2009 047 228 A1 discloses a method of preparing (meth)acrylates from C₁₇ alkanol mixtures by esterification of (meth)acrylic acid with a C₁₇ alkanol mixture in the presence of an acidic catalyst, a polymerization inhibitor and a solvent which forms an azeotrope with water, wherein the esterification is carried out in a reactor equipped with a circulatory evaporator and the azeotrope is distilled off and condensed. The C₁₇ alkanol mixture has a mean degree of branching of from 2.8 to 3.7. Useful acidic catalysts include mineral acids and sulfonic acids, such as sulfuric acid, phosphoric acid, alkylsulfonic acid, arylsulfonic acid and also acidic ion exchangers and zeolites.

In the example, acrylic acid is esterified with a C₁₇ alkanol mixture. This comprises initially charging cyclohexane, heptadecanol having a mean degree of branching of about 3.0, stabilizer (MEHQ), hypophosphorous acid and copper(II) chloride solution and adding acrylic acid. The mixture is heated under an air atmosphere, methanesulfonic acid is added and water is continuously separated off under reflux. The cooled-down reaction solution is washed with sodium chloride solution and aqueous sodium hydroxide solution. The cyclohexane phase is separated off and the solvent is removed under reduced pressure. Heptadecyl acrylate is obtained in 89% yield and >95% purity. A purifying distillation is not carried out since C₁₇ alkyl (meth)acrylates are of too high a molecular weight to be subjected to purifying distillation at acceptable cost.

The esterification of (meth)acrylic acid with alkanols generates not inconsiderable amounts of by-products by Michael addition. Such by-products include di(alkyl (meth)acrylates) for example. These are high boilers with respect to the target product. Alkyl (meth)acrylates of long-chain alkanols can be separated off from these by-products only by vacuum distillation, and when the reacted alkanols have more than a certain number of carbon atoms only by distillation under greatly reduced pressure, so that economic removal of the alkyl (meth)acrylates is no longer possible at all. Moreover, the catalyst employed and the stabilizer also need to be separated off from the product. Provided that the boiling point of the target product is not too high, said target product is generally subjected to a final purifying distillation.

Higher alkyl (meth)acrylates are also obtainable by catalytic transesterification of methyl or ethyl (meth)acrylate with the appropriate long-chain alkanols. The great advantage of transesterification methods is that di(meth)acrylic esters cannot be formed, with the result that a purer product is generally obtained.

A further advantage of transesterification methods is that the excess reactant (meth)acrylate is separated off by distillation and may thus easily be returned to the process while in esterification methods the reactant (meth)acrylic acid is removed by extraction and can be reused only at great expense and inconvenience. Transesterification methods, like esterification methods, are carried out in the presence of a stabilizer (polymerization inhibitor).

DE 2 317 226 A1 discloses a method of preparing (meth)acrylic esters from a mixture of C₁₀-C₁₈ alkanols by transesterification of methyl (meth)acrylate in the presence of titanium alkoxide as catalyst and 2,6-di-tert-butylparacresol (TBC) as stabilizer. This method is carried out in the presence of activated carbon. After the reaction has ended, water is added to hydrolyze the titanium alkoxide to titanium hydroxide which adsorbs onto the activated carbon. The solid is filtered off and the reaction product is subjected to a steam distillation.

WO 2009/080380 discloses a method of preparing methacrylates from C₆-C₂₂ alcohols by transesterification of methyl (meth)acrylate with the appropriate alcohols in the presence of titanium alkoxide as catalyst. Example 1 comprises reacting methyl methacrylate with 2-ethylhexanol in the presence of hydroquinone monomethyl ether (MEHQ) as stabilizer and tetraisopropyl titanate as catalyst. An azeotropic mixture of methanol/methyl methacrylate is distilled off here. Once unconverted methyl methacrylate has been distilled off, the 2-ethylhexyl methacrylate comprising the catalyst is subjected to a purifying distillation under reduced pressure (about 30 mbar). This affords 2-ethylhexyl methacrylate in 99.4% purity.

DE 10 2004 036 930 A1 discloses a method of preparing (meth)acrylates of N-hydroxyalkylated amides by esterification of (meth)acrylic acid or transesterification of methyl (meth)acrylate with the hydroxyalkylated amides in the presence of a heterogeneous catalyst. Useful heterogeneous catalysts include, inter alia, lithium phosphate, potassium phosphate, sodium phosphate, potassium carbonate and sodium carbonate. The heterogeneous catalysts are generally separated off by filtration, electrofiltration, absorption, centrifugation or decantation.

Common to esterification and transesterification methods is that it is necessary to separate off from the target product not only the catalyst and the excess reactant (meth)acrylic acid or (meth)acrylate respectively, but generally also the process stabilizers employed.

(Meth)acrylic esters are polymerizable compounds. Sufficient polymerization inhibition is thus to be ensured in all method steps. Undesired polymerization is a safety issue on account of the large amount of heat liberated.

Examples of suitable process stabilizers are N-oxides (nitroxyl or N-oxyl radicals, i.e., compounds bearing at least one N—O group), for example 4-hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetramethylpiperidine N-oxyl, bis(1-oxyl-2,2,6,6-tetramethylpiperidin-4-yl) sebacate, 4,4′,4″-tris(2,2,6,6-tetramethylpiperidine N-oxyl) phosphite or 3-oxo-2,2,5,5-tetramethylpyrrolidine N-oxyl; mono- or polyhydric phenols which may bear one or more alkyl groups, for example alkylphenols, for example o-, m- or p-cresol (methylphenol), 2-tert-butylphenol, 4-tert-butylphenol, 2,4-di-tert-butylphenol, 2-methyl-4-tert-butylphenol, 2-tert-butyl-4-methyl phenol, 2,6-tert-butyl-4-methylphenol, 4-tert-butyl-2,6-dimethylphenol or 6-tert-butyl-2,4-dimethylphenol; quinones, for example hydroquinone, hydroquinone monomethyl ether, 2-methylhydroquinone or 2,5-di-tert-butylhydroquinone; hydroxyphenols, for example catechol (1,2-dihydroxybenzene) or benzoquinone; aminophenols, for example p-aminophenol; nitrosophenols, for example p-nitrosophenol; alkoxyphenols, for example 2-methoxyphenol (guaiacol, catechol monomethyl ether), 2-ethoxyphenol, 2-isopropoxyphenol, 4-methoxyphenol (hydroquinone monomethyl ether), mono- or di-tert-butyl-4-methoxyphenol; tocopherols, for example α-tocopherol and 2,3-dihydro-2,2-dimethyl-7-hydroxybenzofuran (2,2-dimethyl-7-hydroxycoumaran), aromatic amines, for example N,N-diphenylamine or N-nitrosodiphenylamine; phenylenediamines, for example N,N′-dialkyl-p-phenylenediamine where the alkyl radicals may be identical or different and each consist independently of from 1 to 4 carbon atoms and may be straight-chain or branched, for example N,N′-dimethyl-p-phenylenediamine or N,N′-diethyl-p-phenylenediamine, hydroxylamines, for example N,N-diethylhydroxylamine, imines, for example methyl ethyl imine or methylene violet, sulfonamides, for example N-methyl-4-toluenesulfonamide or N-tert-butyl-4-toluenesulfonamide, oximes such as aldoximes, ketoximes or amide oximes, for example diethyl ketoxime, methyl ethyl ketoxime or salicylaldoxime, phosphorus compounds, for example triphenylphosphine, triphenyl phosphite, triethyl phosphite, hypophosphorous acid or alkyl esters of the phosphorous acids; sulfur compounds, for example diphenyl sulfide or phenothiazine; metal salts, such as copper, manganese, cerium, nickel or chromium salts, for example chlorides, sulfates, salicylates, tosylates, acrylates or acetates, for example copper acetate, copper(II) chloride, copper salicylate, cerium(III) acetate or cerium(III) ethylhexanoate, or mixtures thereof.

Advantageously, oxygen may additionally be used as a polymerization inhibitor.

To further support the stabilization, the reaction may be carried out in the presence of an oxygenous gas, preferably air or a mixture of air and nitrogen (lean air).

Monomers need to be protected from undesired premature polymerization not only during preparation, using process stabilizers, but also during storage, using storage stabilizers. Storage stabilizers employed are generally stabilizers selected from the group of phenols, for example BHT, Topanol A, hydroquinone and MEHQ.

The process stabilizers are generally distinct from the storage stabilizers and cannot remain in the end product but for a few exceptions.

Even when the process stabilizer used is, for example, MEHQ which is also used as storage stabilizer, the concentration in the process is generally distinctly higher than in the subsequent end product but is at least determined by the process, more specifically by process safety, and cannot be chosen freely.

In order that the concentration of the MEHQ in the end product may also be adjusted freely, this stabilizer initially needs to be separated off at the end of the process in order then to be purposively re-added in the desired concentration.

Provided that the boiling point of the target product is not too high, said target product is generally subjected to a final purifying distillation in order also to separate off, inter alia, the stabilizer. However when the reacted alkanols comprise more than a certain number of carbon atoms, alkyl (meth)acrylates of long-chain alkanols can only be distilled under greatly reduced pressure so that economic separating-off by distillation is no longer possible at all.

In the esterification methods a wash with aqueous NaOH may, in the case of high-boiling monomers, be used to remove by extraction a large part of the MEHQ together with the acidic catalysts used and this is described in WO2009/106550 and DE102009047228 A1. The excess (meth)acrylic acid is also removed by extraction here and cannot easily be reused.

Heterogeneous catalysts or catalysts convertible into heterogeneous residues, as employed in the transesterification methods described in DE 2 317 226 A1, DE 10 2004 036 930 A1 and WO2009/080380, are generally separated off by filtration, electrofiltration, absorption, centrifugation or decantation. The (meth)acrylate employed in excess may be separated off by distillation here. In this process, the process stabilizer employed remains in the end product when an economic purifying distillation is not possible on account of the excessively high boiling point.

It is an object of the invention to provide an easily performable and economically viable method of preparing C₈-C₂₂ alkyl (meth)acrylates wherein the C₈-C₂₂ alkyl (meth)acrylates are obtained in high purity without a purifying distillation being carried out. It is a particular object of the invention to provide a method wherein both the heterogeneous catalyst and the stabilizer may be easily separated off from the target product and the reactant (meth)acrylate employed in excess may be easily reused.

The object is achieved by a method of preparing a C₈-C₂₂ alkyl (meth)acrylate by transesterification of C₁-C₂ alkyl (meth)acrylate with a C₈-C₂₂ alkanol, said method comprising the steps of

(i) reacting C₁-C₂ alkyl (meth)acrylate with the C₈-C₂₂ alkanol in the presence of a particulate potassium phosphate heterogeneous catalyst and a stabilizer thus releasing C₁-C₂ alkanol,

(ii) continuously distilling off the azeotrope of C₁-C₂ alkyl (meth)acrylate and C₁-C₂ alkanol,

wherein steps (i) and (ii) are carried out simultaneously until substantially all of the C₈-C₂₂ alkanol has reacted,

(iii) distilling off unconverted C₁-C₂ alkyl (meth)acrylate,

(iv) washing the C₈-C₂₂ alkyl (meth)acrylate-comprising product mixture obtained in steps (i) through (iii) with an aqueous phase to separate off the catalyst and the stabilizer from the product mixture with the aqueous phase and optionally adding a stabilizer,

(v) distilling off water from the product mixture,

wherein step (iii) may also be effected after step (iv) and together with step (v) and step (v) affords a product having a purity of >98 wt %.

It has been found that, surprisingly, transesterification of C₁-C₂ alkyl (meth)acrylate with C₈-C₂₂ alkanols in the presence of a particulate potassium phosphate heterogeneous catalyst allows both the particulate catalyst and the stabilizer to be separated off in a single washing step with water. The product is obtained in at least >98 wt % purity. In the case of linear C₈-C₂₂ alkanols, the product purity is >98 wt %. In the case of branched C₈-C₂₂ alkanols, the product purity is in fact >99 wt %. Purposive readdition of a defined amount of the stabilizer to the product may be carried out subsequently.

One embodiment of the invention comprises reacting methyl (meth)acrylate.

A further embodiment of the invention comprises reacting ethyl (meth)acrylate.

Preferred C₈-C₂₂ alkanols reacted according to the method of the invention are isomer mixtures of C₉ alkanols (isononanol), C₁₀ alkanols, particularly those comprising 2-propylheptanol, isodecanol, lauryl alcohol, C₁₃ alkanols (tridecanols), C₁₇ alkanols (heptadecanols), C₁₆/C₁₈ alkanols, particularly those comprising stearyl alcohol, C₁₈/C₂₂ alkanols, particularly those comprising behenyl alcohol, and C₂₁ alkanols.

2-Propylheptanol is generally employed as a C₁₀ alkanol mixture comprising 2-propylheptanol as the main isomer. The 2-propylheptanol content of this mixture is generally at least 50% by weight, preferably from 60% to 98% by weight, more preferably from 80% to 95% by weight and more particularly from 85% to 95% by weight in each case based on the overall weight of the C₁₀ alkanol mixture.

In addition to comprising 2-propylheptanol as the main isomer, the C₁₀ alkanol mixture generally also comprises at least one of the C₁₀ alcohols selected from the group consisting of 2-propyl-4-methylhexanol, 2-propyl-5-methylhexanol, 2-isopropylhexanol, 2-isopropyl-4-methylhexanol, 2-isopropyl-5-methylhexanol and 2-propyl-4,4-dimethylpentanol. 2-Propylheptanol may be prepared as described in DE 10 2007 001 540 A1.

Preferred C₁₇ alkanol mixtures have a C₁₇ alkanol content of at least 95% by weight, particularly preferably at least 98% by weight and more particularly at least 99% by weight based on the overall weight of the C₁₇ alkanol mixture. Further preferred isomer mixtures of C₁₇ alkanols have a mean degree of branching (iso index) of from 2.8 to 3.7, particularly preferably from 2.9 to 3.6, more particularly from 3.05 to 3.4. The preparation of such C₁₇ alkanol mixtures is described in WO 2009/124979 A1.

The reaction of C₁-C₂ alkyl (meth)acrylate with the C₈-C₂₂ alkanol is effected in the presence of potassium phosphate as catalyst. Said catalyst is generally employed in amounts of from 0.2 to 10 mol %, preferably from 1 to 4 mol %, based on the C₈-C₂₂ alkanol employed.

The reaction of C₁-C₂ alkyl (meth)acrylate with the C₈-C₂₂ alkanol is effected in the further presence of a stabilizer which functions as a polymerization inhibitor. As explained above, the transesterification of (meth)acrylates requires the presence of a process stabilizer.

In the method according to the invention, both the transesterification reaction and the thermal separations are thus preferably carried out in the presence of customary amounts of polymerization inhibitors known per se. The method generally employs one or more suitable process stabilizers in an amount of 10-5000 ppm, preferably 50-5000 ppm and more preferably 100-2000 ppm, based on the total amount of the reaction mixture.

Suitable stabilizers are hydroquinone, hydroquinone monomethyl ether, 2-tert-butylphenol, 4-tert-butylphenol, 2-tert-butyl-4-methylphenol, 6-tert-butyl-2,4-dimethylphenol, 2-methyl-4-tert-butylphenol. Preferred stabilizers are hydroquinone and hydroquinone monomethyl ether (MEHQ), hydroquinone monomethyl ether being particularly preferred.

The transesterification reaction (steps (i) and (ii)) is generally carried out at a temperature of from 60° C. to 140° C., preferably from 70° C. to 110° C. An azeotrope of solvent, C₁-C₂ alkyl (meth)acrylate and C₁-C₂ alkanol is continuously distilled off from the reaction. The azeotropic mixture of C₁-C₂ alkanol and C₁-C₂ alkyl (meth)acrylate is generally distilled off via a suitable column.

The C₁-C₂ alkyl (meth)acrylate is generally employed in a stoichiometric excess. The excess of C₁-C₂ alkyl (meth)acrylate per hydroxyl group to be esterified is preferably from 5 to 1000 mol %, more preferably from 50 to 500 mol % and more particularly from 100 to 300 mol %.

The transesterification may be carried out at atmospheric pressure but also at superatmospheric pressure or reduced pressure. Said transesterification is generally carried out at from 200 to 1000 mbar, preferably from 300 to 700 mbar. The reaction time is generally from 30 minutes to 20 hours, preferably from 1 to 6 hours.

The transesterification (steps (i) and (ii)) may be carried out in any reactor suitable for such a reaction. Such reactors are known to those skilled in the art. The reaction is preferably carried out in a stirred-tank reactor.

The batch may be commixed using any desired method, for example stirring means; commixing may also be achieved by injection of a gas, preferably an oxygenous gas.

The transesterification (steps (i) and (ii)) is generally carried out in a reactor equipped with a distillation column comprising a condenser. The reactor may be a reactor with jacket heating and/or internal heating coils or a reactor having an external heat exchanger and natural or forced circulation (using a pump) may be used. In the case of natural circulation, the cycle stream is accomplished without mechanical aids. The distillation column is of a design known per se and comprises typical internals. Useful column internals include in principle all commonly used internals, for example trays, structured packings or random packings. From 5 to 20 theoretical plates are generally sufficient.

Steps (i) and (ii) are carried out until substantially all of the C₈-C₂₂ alkanol has reacted. This is the case when 98%, preferably 99% and more preferably 99.5% of the C₈-C₂₂ alkanol has reacted.

Unconverted C₁-C₂ alkyl (meth)acrylate is subsequently distilled off from the product mixture in a distillation step (iii). This distillation is effected, for example, in a stirred tank with jacket heating and/or internal heating coils at a temperature of from 40° C. to 100° C., preferably from 40° C. to 80° C., and a pressure of from 1 to 800 mbar, preferably from 10 to 200 mbar, for example via a column as already used in steps (i) and (ii).

It will be appreciated that the distillation may also be carried out in a falling-film or thin-film evaporator. To this end, the reaction mixture is passed through the apparatus, preferably two or more times in a circuit, at reduced pressure, for example at from 20 to 700 mbar, preferably from 30 to 500 mbar and more preferably from 50 to 150 mbar, and a temperature of from 40° C. to 80° C.

An inert gas, preferably an oxygenous gas and more preferably air or a mixture of air and nitrogen (lean air), may advantageously be introduced into the distillation apparatus, for example from 0.1 to 1, preferably from 0.2 to 0.8 and more preferably from 0.3 to 0.7 m³/m³h based on the volume of the reaction mixture.

At least one washing step (iv) is subsequently carried out which comprises contacting with water the C₈-C₂₂ alkyl (meth)acrylate-comprising product mixture which still comprises the stabilizer and the catalyst. It is also possible to carry out two or more washing steps, for example three washing steps. The amount of washing water employed per washing step is generally from 0.1 to 2 times, preferably from 0.2 to 0.5 times, the amount of product mixture.

The wash may be carried out in a stirred vessel for example or in another conventional apparatus, for example in a column or mixer-settler apparatus.

In process engineering terms, a wash in the method according to the invention may be carried out using all extraction and washing methods and apparatuses known per se, for example those described in Ullmann's Encyclopedia of Industrial Chemistry, 6th ed., 1999 Electronic Release, chapter “Liquid—Liquid Extraction—Apparatus”. For example, these may be single- or multistage, preferably single-stage, extractions and also extractions in cocurrent or countercurrent mode.

The washed reaction mixture is optionally admixed with a storage stabilizer such that the target product comprises the desired concentration of stabilizer, for example 100 ppm. This concentration which may be adjusted as desired with this method depends on the particular specification of the end product and for commercial alkyl (meth)acrylates is in the range of from 15 to 200 ppm for example. The storage stabilizers are generally stabilizers selected from the group of phenols, for example 2,6-di-tert-butyl-4-methylphenol, 6-tert-butyl-2,4-dimethylphenol, hydroquinone and hydroquinone monomethyl ether, preferably hydroquinone monomethyl ether.

Residual water is subsequently distilled off from the product mixture in a further distillation step (v). This distillation is generally effected at a temperature of from 40° C. to 100° C., preferably from 40° C. to 80° C., and a pressure of from 1 to 800 mbar, preferably from 10 to 300 mbar, for example via a column as already used in steps (i) and (ii).

It will be appreciated that the distillation may also be carried out in a falling-film or thin-film evaporator. To this end, the reaction mixture is passed through the apparatus, preferably two or more times in a circuit, at reduced pressure, for example at from 20 to 700 mbar, preferably from 30 to 500 mbar and more preferably from 50 to 150 mbar, and a temperature of from 40° C. to 80° C.

An inert gas, preferably an oxygenous gas and more preferably air or a mixture of air and nitrogen (lean air), may advantageously be introduced into the distillation apparatus, for example from 0.1 to 1, preferably from 0.2 to 0.8 and more preferably from 0.3 to 0.7 m³/m³h based on the volume of the reaction mixture.

Optionally, distillation step (iii) may also be effected together with step (v).

Once distillation (v) has been carried out there remains a product obtained in the purity described hereinabove.

The invention is more particularly described using the examples which follow.

### EXAMPLES

### Example 1

### Transesterification of MMA with Isononanol

The transesterification was effected in a 4 L jacketed reactor furnished with an anchor stirrer, an air inlet, a separating column and a liquid divider. The reflux ratio was variably adjusted to from 99:1 to 5:1 (reflux:distillate) to match the amount of distillate, the stirrer speed was 160 rpm and the air introduction rate was 1.5 L/h.

This apparatus was initially charged with 0.29 g of methylhydroquinone (MEHQ) and 1400 g of methyl methacrylate (MMA, stabilized with 15 ppm of MEHQ) at room temperature. 1010 g of Nonanol N (CAS: 27458-94-2, isomer mixture, iso index 1.2) and 29.7 g of potassium phosphate were added and the reaction mixture was heated up at a bath temperature of 90° C.

A pressure of 350 mbar (abs.) was established and an azeotrope of methanol and MMA was continuously distilled off while the bottoms temperature increased from 68° C. to 88° C. The bath temperature was adjusted to 100° C. toward the end. On termination of the reaction, excess MMA was distilled off under reduced pressure and the bath temperature was reduced to 60° C. Once cooled down to room temperature the product was washed 3× with 500 mL of water each time and each of the aqueous phases was separated off and discarded. 120 mg of MEHQ were added, the residual water was distilled off under reduced pressure and the mobile product was filtered using a paper filter.

The product isononyl methacrylate was obtained in a yield of 1474 g (99%) in >99% purity. The MEHQ content was 120 ppm.

### Example 2

### Transesterification of MMA with Tridecanol N

The transesterification was effected in a 4 L jacketed reactor furnished with an anchor stirrer, an air inlet, a separating column and a liquid divider. The reflux ratio was variably adjusted to from 99:1 to 5:1 (reflux:distillate) to match the amount of distillate, the stirrer speed was 160 rpm and the air introduction rate was 1.5 L/h.

This apparatus was initially charged with 0.31 g of methylhydroquinone (MEHQ) and 1600 g of methyl methacrylate (MMA, stabilized with 15 ppm of MEHQ) at room temperature. 986 g of Tridecanol N (isomer mixture, iso index 2) and 17 g of potassium phosphate were added and the reaction mixture was heated up at a bath temperature of 90° C.

A pressure of 300 mbar (abs.) was established and an azeotrope of methanol and MMA was continuously distilled off while the bottoms temperature increased from 69° C. to 77° C. The bath temperature was adjusted to 100° C. toward the end. On termination of the reaction, excess MMA was distilled off under reduced pressure and the bath temperature was reduced to 50° C. Once cooled down to room temperature the product was washed 3× with 400 mL of water each time and each of the aqueous phases was separated off and discarded. 110 mg of MEHQ were added, the residual water was distilled off under reduced pressure and the product was filtered using a pressure filter (1.5 bar).

The product tridecyl methacrylate was obtained in a quantitative yield of 1335 g in >99% purity. The MEHQ content was 90 ppm.

### Example 3

### Transesterification of MMA with C₁₇ Alcohol

The transesterification was effected in a 4 L jacketed reactor furnished with an anchor stirrer, an air inlet, a separating column and a liquid divider. The reflux ratio was variably adjusted to from 99:1 to 1:10 (reflux:distillate) to match the amount of distillate, the stirrer speed was 160 rpm and the air introduction rate was 1.5 L/h.

This apparatus was initially charged with 0.31 g of methylhydroquinone (MEHQ) and 1600 g of methyl methacrylate (MMA, stabilized with 15 ppm of MEHQ) at room temperature. 986 g of Heptacanol N (isomer mixture, iso index 3) and 17 g of potassium phosphate were added and the reaction mixture was heated up at a bath temperature of 100° C.

A pressure of 300 mbar (abs.) was established and an azeotrope of methanol and MMA was continuously distilled off while the bottoms temperature increased from 70° C. to 81° C.

On termination of the reaction, excess MMA was distilled off under reduced pressure and the bath temperature was reduced to 50° C. Once cooled down to room temperature the product was washed 3× with 500 mL of water each time and each of the aqueous phases was separated off and discarded. 110 mg of MEHQ were added, the residual water was distilled off under reduced pressure and the product was filtered using a pressure filter (1.5 bar).

The product heptadecyl methacrylate was obtained in a yield of 1210 g (93%) in >99.5% purity. The MEHQ content was 110 ppm.

### Example 4

### Transesterification of MMA with C₂₁ Alcohol

The transesterification was effected in a 0.75 L jacketed reactor furnished with an anchor stirrer, an air inlet, a separating column and a liquid divider. The reflux ratio was variably adjusted to from 99:1 to 1:10 (reflux:distillate) to match the amount of distillate, the stirrer speed was 160 rpm and the air introduction rate was 1.5 L/h.

This apparatus was initially charged with 0.11 g of methylhydroquinone (MEHQ) and 500 g of methyl methacrylate (MMA, stabilized with 15 ppm of MEHQ) at room temperature. 391 g of heneicosanol (isomer mixture, iso index 3) and 10.6 g of potassium phosphate were added and the reaction mixture was heated up at a bath temperature of 100° C.

A pressure of 300 mbar (abs.) was established and an azeotrope of methanol and MMA was continuously distilled off while the bottoms temperature increased from 71° C. to 81° C. On termination of the reaction, excess MMA was distilled off under reduced pressure and the bath temperature was reduced to 65° C. Once cooled down to room temperature the product was washed 2× with 300 mL of water each time and each of the aqueous phases was separated off and discarded. 40 mg of MEHQ were added, the residual water was distilled off under reduced pressure and the product was filtered using a pressure filter (1.5 bar).

The product heneicosyl methacrylate was obtained in a yield of 1210 g (93%) in >99.8% purity. The MEHQ content was 120 ppm.

### Example 5

### Transesterification of MMA with Stearyl Alcohol

The transesterification was effected in a 0.75 L jacketed reactor furnished with an anchor stirrer, an air inlet, a separating column and a liquid divider. The reflux ratio was variably adjusted to from 99:1 to 1:10 (reflux:distillate) to match the amount of distillate, the stirrer speed was 250 rpm and the air introduction rate was 1.5 L/h.

This apparatus was initially charged with 0.26 g of methylhydroquinone (MEHQ) and 300 g of methyl methacrylate (MMA, stabilized with 15 ppm of MEHQ) at room temperature. 262 g of stearyl alcohol (C₁₆/C₁₈ 0.3/0.7) and 8.5 g of potassium phosphate were added and the reaction mixture was heated up at a bath temperature of 110° C.

A pressure of 600 mbar (abs.) was established and an azeotrope of methanol and MMA was continuously distilled off while the bottoms temperature increased from 87° C. to 94° C. On termination of the reaction the product was cooled down to 40° C., washed 3× with 150 mL of water each time and each of the aqueous phases was separated off and discarded. 30 mg of MEHQ were added, excess MMA was distilled off under vacuum with the residual water at a bath temperature of 80° C. and the product was filtered while warm using a pressure filter (1.5 bar).

The product stearyl methacrylate was obtained in a quantitative yield of 328 g in >98% purity. The MEHQ content was 110 ppm.

### Comparative Examples

### (Transesterification with Filtration)

Comparative Example 1 Transesterification of MMA with Tridecanol N with filtration

The transesterification was effected in a 4 L jacketed reactor furnished with an anchor stirrer, an air inlet, a separating column and a liquid divider. The reflux ratio was 5:1 (reflux: distillate), the stirrer speed was 180 rpm and the air introduction rate was 1.5 L/h.

This apparatus was initially charged with 0.36 g of methylhydroquinone (MEHQ) and 2000 g of methyl methacrylate (MMA, stabilized with 15 ppm of MEHQ) at room temperature. 1000 g of Tridecanol N (isomer mixture, iso index 2) and 21.2 g of potassium phosphate were added and the reaction mixture was heated up at a bath temperature of 90° C.

A pressure of 300 mbar (abs.) was established and an azeotrope of methanol and MMA was continuously distilled off while the bottoms temperature increased from 69° C. to 77° C. The bath temperature was adjusted to 100° C. toward the end. On termination of the reaction the residual MMA was distilled off under reduced pressure and the bottoms temperature was reduced to 50° C. The product was filtered using a paper filter.

The product tridecyl methacrylate was obtained in a quantitative yield of 1323 g (99%) in >99% purity. The MEHQ content was 200 ppm.

### Comparative Example 2

### Transesterification of MMA with Stearyl Alcohol with Filtration

The transesterification was effected in a 4 L jacketed reactor furnished with an anchor stirrer, an air inlet, a separating column and a liquid divider. The reflux ratio was variably adjusted to from 99:1 to 1:1 (reflux:distillate) to match the amount of distillate, the stirrer speed was 160 rpm and the air introduction rate was 2.5 L/h.

This apparatus was initially charged with 0.39 g of methylhydroquinone (MEHQ) and 2003 g of methyl methacrylate (MMA, stabilized with 15 ppm of MEHQ) at room temperature. 1282 g of stearyl alcohol (C₁₆/C₁₈ 0.3/0.7) and 31.8 g of potassium phosphate were added and the reaction mixture was heated up at a bath temperature of 110° C.

A pressure of 400 mbar (abs.) was established and an azeotrope of methanol and MMA was continuously distilled off while the bottoms temperature increased from 77° C. to 84° C. On termination of the reaction the reaction mixture was filtered using a pressure filter (1.5 bar).

The residual MMA was distilled off at reduced pressure at a bath temperature of 60° C.

The product stearyl methacrylate was obtained in a yield of 1572 g (97%) in >98% purity. The MEHQ content was 250 ppm.

1. The invention of preparing a C₈-C₂₂ alkyl (meth)acrylate by transesterification of C₁-C₂ alkyl (meth)acrylate with a C₈-C₂₂ alkanol, said method comprising the steps of
(i) reacting C₁-C₂ alkyl (meth)acrylate with the C₈-C₂₂ alkanol in the presence of a particulate potassium phosphate heterogeneous catalyst and a stabilizer thus releasing C₁-C₂ alkanol,
(ii) continuously distilling off the azeotrope of C₁-C₂ alkyl (meth)acrylate and the C₁-C₂ alkanol,
wherein steps (i) and (ii) are carried out simultaneously until substantially all of the C₈-C₂₂ alkanol has reacted,
(iii) distilling off unconverted C₁-C₂ alkyl (meth)acrylate,
(iv) washing the C₈-C₂₂ alkyl (meth)acrylate-comprising product mixture obtained in steps (i) through (iii) with an aqueous phase to separate off the catalyst and the stabilizer from the product mixture with the aqueous phase and optionally adding a stabilizer,
(v) distilling off water from the product mixture,
wherein step (iii) may also be effected after step (iv) and together with step (v) and step (v) affords a product having a purity of >98 wt %.

2. The method according to claim 1 wherein the C₈-C₂₂ alkanol reacted in the method according to the invention is selected from the group consisting of isomer mixtures of C₉ alkanols, C₁₀ alkanols, isodecanol, lauryl alcohol, C₁₃ alkanols, C₁₇ alkanols, C₁₆/C₁₈ alkanols, C₁₈/C₂₂ alkanols and C₂₁ alkanols.

3. The method according to claim 1 wherein the stabilizer is methylhydroquinone.

4. The method according to claim 1 wherein the C₁-C₂ alkyl (meth)acrylate employed is methyl (meth)acrylate.

5. The method according to claim 1 wherein the C₁-C₂ alkyl (meth)acrylate employed is ethyl (meth)acrylate.
